(19) United States
(12) Patent Application Publication
(10) Pub. No.: US 2014/0234226 A1
(43) Pub. Date: Aug. 21, 2014

(54) PLASMONIC STABLE FLUORESCENCE SUPERPARAMAGNETIC IRON OXIDE NANOPARTICLES AND A METHOD OF SYNTHESIZING THE SAME

(71) Applicants: Morteza Mahmoudi, Tehran (IR); Mohammad Ali Shokrgozar, Tehran (IR)

(72) Inventors: Morteza Mahmoudi, Tehran (IR); Mohammad Ali Shokrgozar, Tehran (IR)

(21) Appl. No.: 14/270,879

(22) Filed: May 6, 2014

(51) Int. Cl. A61K 49/18

(52) U.S. Cl. CPC A61K 49/1863
USPC 424/9.323

(57) ABSTRACT

The various embodiments herein provide for the engineered multimodal super paramagnetic iron oxide nanoparticles (SPIONs) with a fluorescent dye. The SPIONs comprise fluorescent polymer dye arranged in a gap between a SPION core and a gold shell. The SPIONS are provided with a gold coating. The gap is made up of a polymeric molecule such as 6-arm anthracene terminated. The core of the nanoparticle is made up of a magnetic metal oxide. The method for synthesizing SPIONs involves preparing carboxyl-dextran complex and the SPIONS. The SPIONs are coated with carboxyl-dextran complex. The coated SPIONs coated are subjected to fluorescent polymer and gold nano shell coating. The prepared SPIONs are characterized by light scattering measurement and magnetization measurements.

### BACKGROUND

1. Technical Field

The embodiments herein generally relate to a field of molecular imaging and multimodal molecular imaging in a medical theranosis. The embodiments herein generally relate the multifunctional engineered nanoparticles with desired bio-physio chemical properties. The embodiments herein more particularly relate to gold coated super paramagnetic iron oxide nanoparticles (SPIONs) with fluorescence capability and a method of synthesizing the gold coated super paramagnetic iron oxide nanoparticles (SPION) with a fluorescent layer.

2. Description of the Related Art

With the application of nanotechnology in the medical field, one can hope for early diagnosis together with a prompt treatment of catastrophic diseases like cancer. The treatment of these catastrophic diseases has been dramatically increased with the application of nanoscience in medical treatment. Development of multifunctional engineered nanoparticles (NPs) with desired physiochemical properties, as nano-probes, has enabled new imaging modalities to provide a great capability in molecular imaging and medical theranosis and which are essential for an early detection and a rapid treatment of the diseases. It is noteworthy to mention that these multimodalities of engineered nanoparticles (NPs) are beyond the observed intrinsic properties of materials comprising individual NPs. In the past few decades, nanoparticles (NPs) have been recognized as promising candidates for creating a new revolution in science and technology due to their unusual properties which have attracted the attention of physicists, chemists, biologists and engineers. The application of NPs in medical sciences either introduced new opportunities or caused significant enhancements in the conventional biomedical methods such as imaging purposes. The creation of novel engineered multimodal NPs is a key focus in bio-nanotechnology and can lead to an advancement in the deep understanding of the biological processes at a bio-molecular level thereby causing a great impact on the molecular diagnostics, imaging and therapeutic applications.

Ever since the medical diagnosis era was initiated by Wilhelm Roentgen, who captured the first X-ray image of his wife's hand in 1896, X-rays have been extensively employed in the medical imaging of anatomical details. However, cellular and molecular imaging still remained as dreams in the field. With the development of nano science, this dream has come true. The advantage of using the multimodal NPs, in comparison with the individual NPs such as semiconductor quantum dots, magnetic and metallic NPs, for cellular or biomolecular tracking, is the capability of multimodal NPs to provide a high spatial resolution with high contrast for an anatomic background, together with the lack of exposure to ionizing radiation and the ability to follow the cells for months. With the successful introduction of nanoscience and nanotechnology in the medical field, one can hope for fast theranosis of catastrophic diseases like cancer and diabetes, in a recent future. Development of new multifunctional engineered nanoparticles with desired bio-physio chemical properties can enable new imaging modalities which have a great capability in molecular imaging and medical theranosis, which are essential for an early detection and a rapid treatment of diseases.

Super paramagnetic iron oxide nanoparticles (SPIONs) are an emerging form of nano-medicine for the treatment and diagnosis of diseases. Several requirements must be met for SPIONs before being successfully used in the treatment and diagnosis. The SPIONs must be completely dispersed in physiological medium. Secondly the SPIONs must be biocompatible and the SPIONs must have a capability to prevent an adsorption of plasma proteins or cells onto their surface.

The nano particles used in the area of nanomedicine for therapeutics and diagnostics include quantum dots, metallic nanoparticles and magnetic nanoparticles etc.

The quantum dots are used for the diagnosis of tumors and metabolic malfunctions. The use of quantum dots for diagnosis and therapeutics becomes difficult because of their large size (10-30 nm). Secondly the quantum dots are known to have “blinking behavior”, wherein the dark periods persist without any emission from quantum dots. The dark periods interrupt longer periods of fluorescence, thereby making the diagnosis or therapy difficult.

The metallic nanoparticles are also used for therapeutic and diagnostic applications. The metallic nanoparticles pose a threat because of their pyrophoric and reactive properties. The metallic nanoparticles are reactive towards oxidizing agents. Because of the reactive and pyrophoric properties, the metallic nanoparticles are difficult to handle. Some nanoparticles, such as silver nanoparticles, have shown toxic effect. The silver nanoparticles are well recognized as the promising antimicrobial agents among the various types of nanoparticles. However there are two major shortcomings with these particles. Firstly, the silver nanoparticles have a toxic effect on the human cells and secondly the silver nanoparticles have a low yield for penetration through the bacterial bio-films.

The magnetic nanoparticles are also used for therapeutic and diagnostic applications. The magnetic nanoparticles are reactive towards the oxidizing agents. The reactivity of the magnetic nanoparticles make them unfit to be used alone in therapeutic and diagnostic applications.

According to one prior art, several efforts were made to attach fluorescence dyes to the surface of nanoparticles. But the cells act as extremely efficient filters for the elution of the surface bounded fluorescent tags with nanoparticles. Secondly, the fluorescent capability encounters a significant decay after a short period of time. The reason for the decay in the fluorescent capability of nanoparticles is due to the fact that the surface fluorescent tag is in direct contact with cellular fluids.

Hence there is a need to develop super paramagnetic iron oxide nanoparticles (SPIONs) with permanent fluorescence capability for diagnostic and therapeutic applications. Also there is a need develop super paramagnetic iron oxide nanoparticles (SPIONs) with permanent fluorescence capability for use as nano probes in molecular imaging and invitro tracking purposes.

The above mentioned shortcomings, disadvantages and problems are addressed herein and which will be understood by reading and studying the following specification.

### OBJECTIVES OF THE EMBODIMENTS

The primary object of the embodiments herein is to provide multimodal plasmonic super paramagnetic iron oxide nanoparticles (SPIONs) with the permanent fluorescence capability for use in Molecular diagnostics, imaging and therapeutic applications.

Another object of the embodiments herein is to provide multimodal bio-imaging super paramagnetic iron oxide nanoparticles (SPIONs) with permanent fluorescence capability for a deep understanding of the biological processes at bio-molecular level.

Yet another object of the embodiments herein is to provide super paramagnetic iron oxide nanoparticles (SPIONs) with the permanent fluorescence capability for molecular diagnostics, imaging and therapeutic applications.

Yet another object of the embodiments herein is to provide super paramagnetic iron oxide nano particles (SPIONs) with the permanent fluorescence capability for early an detection and rapid treatment of diseases.

Yet another object of the embodiments herein is to provide super paramagnetic iron oxide nanoparticles (SPIONs) with the permanent fluorescence capability and enhanced contrast specificity.

Yet another object of the embodiments herein is to provide super paramagnetic iron oxide nanoparticles (SPIONs) having a combination of gold and magnetic nanoparticles with the permanent fluorescence capability.

Yet another object of the embodiments herein is to provide super paramagnetic iron oxide nanoparticles (SPIONs) with the permanent fluorescence capability, controllable shell thickness and smooth surface.

Yet another object of the embodiments herein is to provide super paramagnetic iron oxide nanoparticles (SPIONs) with a polymeric dielectric layer in a gap between a magnetic core and smooth gold nano shell.

Yet another object of the embodiments herein is to provide super paramagnetic iron oxide nanoparticles (SPIONs) with a fluorescent polymorphic dye trapped between the magnetic core and gold nanoshell.

Yet another object of the embodiments herein is to provide super paramagnetic iron oxide nanoparticles (SPIONs) with uniform gold coating and permanent fluorescence capability.

Yet another object of the embodiments herein is to provide super paramagnetic iron oxide nanoparticles (SPIONs) with the permanent fluorescence capability, which are magnetically sensitive to Near-Infrared Spectroscopy (NIR), Magnetic Resonance Imaging (MRI), Magnetomotive Photoacoustic Imaging (mmPA).

Yet another object of the embodiments herein is to provide iron oxide nanoparticles (SPIONs) with the permanent fluorescence capability to allow deep tissue imaging along with magnetic resonance imaging (MRI).

Yet another object of the embodiments herein is to provide super paramagnetic iron oxide nanoparticles (SPIONs) with enhanced contrast specificity in diagnostics.

Yet another object of the embodiments herein is to provide super paramagnetic iron oxide nanoparticles (SPIONs) with fluroscence capability and without any chance for fluroscence dilution thereby enhancing the capability of the nanoprobes for molecular imaging and in vitro/invivo tracking purposes.

These and other objects and advantages of the embodiments herein will become readily apparent from the following detailed description taken in conjunction with the accompanying drawings.

### SUMMARY

The embodiments herein provide the engineered multimodal super paramagnetic iron oxide nanoparticles (SPIONs) with a fluorescent dye and a method of synthesizing the SPIONs.

According to one embodiment herein, the engineered multimodal nanoparticles with a fluorescent dye comprise a super paramagnetic iron oxide nanoparticle (SPION) with at least one coating and at least one gap. The coating is made up of a metal. The gap is made up of a polymeric molecule. The core of the nanoparticle is made up of oxide of a magnetic metal.

According to one embodiment herein, the super paramagnetic iron oxide nanoparticles (SPIONs) comprises a dextran with an average molecular weight of 5000, sodium periodate, potassium cyanide, poly-L-histidine with a molecular weight of 5000-25000, and poly (ethylene oxide), 6-arm anthracenes with an average molecular weight of 12000, 90% oleic acid, 1-octadecene, oleyl alcohol and n-hexane.

According to an embodiment herein, a plasmonic stable fluorescence super paramagnetic iron oxide nanoparticle (SPION) is provided. The plasmonic stable fluorescence super paramagnetic iron oxide nanoparticle (SPION) comprises a nano metal core and wherein the nano metal core is formed with a SPION. A nano shell is arranged around the nano metal core and wherein the nano shell is a gold shell. A dielectric polymer layer is formed in a gap between the nano metal core and the nano shell, and wherein the dielectric polymer layer is a fluorescence polymer layer. The nano metal core is made up of ferrous chloride. The fluorescent polymer is 6-arm anthracene terminated. The SPION has a particle size of 13 mm.

According to an embodiment herein, a method for synthesizing plasmonic stable fluorescence super paramagnetic iron oxide nanoparticles (SPIONs) is provided. The method comprises the steps of preparing carboxylated dextran, preparing super paramagnetic iron oxide nanoparticle (SPION), preparing carboxylated dextran coated SPION and preparing a gold coated SPION with fluorescence polymeric gap.

According to an embodiment herein, the step of preparing carboxyl-dextran comprises dissolving sodium periodate in deoxygenated distilled water and wherein an amount sodium periodate dissolved in deoxygenated distilled water is 4 gm, and wherein an amount of deoxygenated distilled water used for dissolving 4 gm of sodium periodate is 30 ml. Dextran solution is added to the solution of sodium periodate. The solution of periodate added with dextrin is homogenized for 2 hrs at room temperature. The homogenized solution is dialyzed in a membrane bag for 4 days and wherein the membrane bag has a cut-off molecular weight of 1,000. A cyanohydrin intermediate is prepared by interacting the dialyzed solution with potassium cyanide. A carboxylated dextran is obtained by a hydrolysis of the intermediate cyanohydrins. The carboxylated dextran is lyophilized at −80° C. and the carboxylated dextran which is lyophilized, is stored.

According to an embodiment herein, the step of preparing SPION comprises dissolving iron oleate complex and 1-octadecene in oleic acid at room temperature to obtain a reaction mixture, wherein an amount of iron oleate complex dissolved is 18 gm, and wherein an amount of iron oleic acid used for dissolving is 5.7 gm, and wherein an amount of 1-octadecene dissolved is 100 gm, and wherein a molarity of the reaction mixture is 20 mmol. The reaction mixture is degassed at 80° C. for 2 hrs. The reaction mixture is heated to a reflux temperature at a rate of 3° C./min. The reaction mixture is incubated for 30 min under an inert atmosphere. The reaction mixture is rapidly cooled to a room temperature. Acetone is added to the cooled reaction mixture and the amount acetone added to the cooled reaction mixture is 500 ml. The SPIONs are precipitated and the SPIONs are separating by centrifugation. The separated SPIONs are dispersed in hexane and the concentration of SPIONS dispersed in hexane is 1 mg/ml.

According to an embodiment herein, the step of synthesizing the carboxyl-dextran coated SPIONs comprises mixing SPION stock solution with dextran, in dimethyl sulfoxide (DMSO), and wherein an amount of SPION stock solution mixed with dimethyl sulfoxide (DMSO) is 1 ml, and wherein an amount of dimethyl sulfoxide (DMSO) mixed with SPION stock solution is 30 ml. The SPIONs are magnetically collected through a strong magnetic field using a magnetically activated cell sorter (MACS®) system and the collected SPIONs are redispersed into 1 ml of distilled water.

According to an embodiment herein, the step of synthesizing the gold coated SPIONs with fluorescent polymeric gap comprises mixing carboxyl-dextran coated SPIONs with poly(ethylene oxide) for 10 hrs and 6-arm anthracene terminated in distilled water using a shaking incubator. The coated SPIONs are collected with a strong magnet. Poly-L histidine is added to a solution of the SPIONs. A pH of a solution of SPIONS and poly-L histidine is adjusted using 0.1N HCl, and wherein the pH of the solution of SPIONS is adjusted to be within 5-6. The pH adjusted solution of SPIONs is incubated for 60 min. The magnetic SPIONS are collected using a magnet after incubating the SPIONs for 60 minutes. The incubated SPIONS are washed with distilled water for several times. A solution of incubated SPIONS is mixing with HAuCl₄ (w/w 1%) and kept for 20 min. A pH of incubated SPIONS solution mixed with HAuCl₄, is adjusted to be in the range of 9-10 using NaOH. A solution of NH₂OH HCl is added to the solution of SPIONS and mixing the solution of SPIONS to obtain a colloidal suspension, wherein the solution of NH₂OH HCl is added to the solution of SPIONS till a color of the colloidal suspension turns to dark blue color. The colloidal suspension is washed several times with distilled water and a colloid is suspended in distilled water. The suspended colloid is incubated in a sonicator at 2-8° C.

According to one embodiment herein, the synthesis of the super paramagnetic iron oxide nanoparticles (SPIONs) is carried out in the following sequences. At first the carboxylated-dextran is prepared. Secondly, the super paramagnetic iron oxide nanoparticles (SPIONs) are prepared. Thirdly, the super paramagnetic iron oxide nanoparticles (SPIONs) are coated with carboxyl-dextran. Then the super paramagnetic iron oxide nanoparticles (SPIONs) coated with carboxyl-dextran coating are further subjected to gold coating Finally, the gold coated, fluorescent gap bearing the super paramagnetic iron oxide nanoparticles (SPIONs) are characterized.

A standard protocol is followed for the preparation of the carboxylated-dextran. For the preparation of carboxylated-dextran, the hydroxyl groups in the dextran are oxidized to aldehyde groups by sodium periodate. Further, sodium periodate is dissolved in the de-oxygenated distilled water and introduced to dextran. An amount of 4 gm of sodium periodate is dissolved in 30 ml of de-oxygenated distilled water. The obtained solution is homogenized for 2 hrs at a room temperature and the homogenized solution is dialyzed using a membrane bag with a cut-off molecular weight of 1,000 for 4 days. Then the obtained solution is subjected to potassium cyanide for the preparation of intermediate cyanohydrins. Finally, the carboxylic acid group is created on the terminal units of dextran by the hydrolysis of the obtained intermediate cyanohydrins. Further, the prepared carboxylated dextran is lyophilized and stored at −80° C.

According to one embodiment herein, a polyol route is employed to obtain the nanoparticles with a narrow size distribution. The method of preparation of SPIONs involves the following sequences. The iron-oleate complexes are prepared by reacting sodium oleate and iron (III) chloride. For the synthesis of SPIONs with a particle size of 13 nm, 18 gm (20 mmol) of iron oleate complex and 5.7 gm of oleic acid (20 mmol) are dissolved in 100 gm of 1-octadecene at room temperature to obtain a reaction mixture. The reaction mixture is degassed at 80° C. temperature for 2 hrs. The reaction mixture is heated to a reflex temperature at a heating rate of 3° C./min. The reaction mixture is then kept for 30 min under inert atmosphere. After the reaction, the reaction vessel is repeatedly cooled at room temperature and 500 mL of acetone is added to precipitate the SPIONs. The SPIONs are separated by the centrifugation and dispersed in hexane.

According to one embodiment herein, the super paramagnetic iron oxide nanoparticles (SPIONs) are coated with carboxyl-dextran. The ligand exchange process is used for coating the prepared hydrophobic nanoparticles with carboxyl-dextran. The SPIONs with an iron concentration of 1 mg/ml is prepared and mixed with dextran ligands in a di-methyl sulfoxide (DMSO). The DMSO is a dipolar solvent and the reactions of nanoparticles and polymers are carried out at room temperature for 72 hrs, while shaking with a shaking incubator. The DMSO is used to form a homogenous solution with both aqueous polymer solution and organic solvent. Specifically, 1 ml of the stock SPION solution is mixed with dextran, in 30 ml of DMSO. After the completion of the reaction, the SPIONs are magnetically collected by a strong magnetic field using a magnetic activated cell sorter (MACS™) system. Further the collected SPIONs are dispersed into 1 ml of distilled or deionized (DI) water. The water soluble SPIONs are completely stable at room temperature.

According to one embodiment herein, the super paramagnetic iron oxide nanoparticles (SPIONs) coated with carboxyl-dextran coating are further subjected to gold coating. The SPIONs are subjected to gold coating to create a fluorescent polymeric gap. The SPIONs are coated with gold to get a smooth surface. The smooth gold-shell SPIONs are prepared by mixing the carboxyl-dextran coated SPIONs with poly (ethylene oxide) and 6-arm anthracene terminated in distilled water using shaking incubator for 10 hrs. The resultant materials after mixing are collected with strong magnet and washed with distilled water for several times. Poly-L histinde (PLH) is added to the solution of SPIONs and the pH of the solution is adjusted to be in the range of 5-6, using 0.1N hydrochloric acid (HCl). After incubating for 60 min, the magnetic nanoparticles are collected with a magnet and washed for several times with distilled water. The obtained solution is mixed with HAuCl₄ (w/w 1%), for 20 min. The pH of the solution is adjusted to be in the range of 9-10 with sodium hydroxide (NaOH). The NH₂OH and HCl is added to the solution and mixed well till the color of the colloidal suspension turns to a dark blue color. The solution is washed for several times, redispersed in distilled water using sonicator and kept at 2-8° C.

According to one embodiment herein, the gold coated, fluorescent gap bearing super paramagnetic iron oxide nanoparticles (SPIONs) are characterized. The first method of characterization is dynamic light scattering (DLS) measurement. The DLS measurement is conducted with a Malvern PCS-4700 instrument equipped with a 256-channel correlator. The 488.0 nm line of a Coherent Innova-70 Ar ion laser is used as the incident beam. The power of the laser used is 250 mW. The scattering angle 0, employed is in the range of 40°-140°. The temperature is maintained at 25° C. with an external circulator. Further, the data obtained is subjected to data analysis and interpretation. Data analysis is performed according to standard procedures, and interpreted through a cumulated expansion of the field auto correlation function of the second order. A constrained regularization method CONTIN is applied to invert the experimental data to obtain a distribution of the decay rates. The size and shape of the nanoparticles are evaluated using a Phillips CM200 transmission electron microscope (TEM) equipped with an AMT 2×2 CCD camera with an accelerating voltage of 200 kV. The sample for TEM is prepared by placing and drying a drop of the suspension on a copper grid.

According to one embodiment herein, the fluorescent gap bearing super paramagnetic iron oxide nanoparticles (SPIONs) are further subjected to magnetization measurements. The solid dry powder of the SPION sample is taken and subjected to Quantum Design Superconducting Quantum Interference Device (SQUID) MPMS-XL7 magnetometer. A hysteresis experiment is performed in the range of −5 T≦H≦+5T at T=300K. The in vitro MRI experiments are performed at 8.5 MHz using a 0.2 Tesla Artoscan Imager by Esaote S.p.A. A Spin Echo (SE) T₂ pulse sequence with the imaging parameters of TR/TE/NEX=2000 ms/80 ms/1, a matrix=256*192, and a FOV=180*180 is used.

According to one embodiment herein, the analysis of super paramagnetic iron oxide nanoparticles (SPIONs) exhibit multimodal imaging. The gold shell bearing SPIONs are compared with the bare SPIONs. The results reveal that the smooth, gold shell coated SPIONs possess both strong scattering property of gold nano shell and fluorescence capability of polymeric gap. Both the properties make the gold coated and fluorescent gap bearing SPIONs as a useful dual-optical imaging probe.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a schematic diagram indicating a structure of gold coated super paramagnetic iron oxide nanoparticles (SPIONs) with stable fluorescence property, according to one embodiment herein.

FIG. 2 illustrates a flow chart explaining a method for the synthesis of super paramagnetic iron oxide nanoparticles (SPIONs) with fluorescent dye, according to one embodiment herein.

FIG. 3 illustrates a schematic diagram indicating a method for the synthesis of super paramagnetic iron oxide nanoparticles (SPIONs) with a fluorescence polymeric gap, according to one embodiment herein.

FIG. 4A illustrates a transmission electron microscope (TEM) image indicating the dark filed imaging of bare SPIONS, according to one embodiment herein.

FIG. 4B illustrates a transmission electron microscope (TEM) image indicating the dark field imaging of smooth gold coated SPIONS with a fluorescence polymeric gap exhibiting the merged fluorescence and scattering images, according to one embodiment herein.

FIG. 4C illustrates a graphical plot indicating a relationship between an amount of magnetization and magnetic field at 300 K for bare super paramagnetic iron oxide nanoparticles (SPIONs) and smooth shaped gold coated SPIONS with fluorescence polymeric gap, according to one embodiment herein.

FIG. 4D illustrates magnetic resonance imaging (MRI) of vials containing bare SPIONS and smooth gold shaped SPIONS with different iron concentrations (SPIONs), according to the embodiments herein.

Although the specific features of the embodiments herein are shown in some drawings and not in others. This is done for convenience only as each feature may be combined with any or all of the other features in accordance with the embodiments herein.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide the engineered multimodal super paramagnetic iron oxide nanoparticles (SPIONs) with a fluorescent dye and a method of synthesizing the SPIONs.

According to one embodiment herein, the engineered multimodal nanoparticles with a fluorescent dye comprise a super paramagnetic iron oxide nanoparticle (SPION) with at least one coating and at least one gap. The coating is made up of a metal. The gap is made up of a polymeric molecule. The core of the nanoparticle is made up of oxide of a magnetic metal.

According to one embodiment herein, the super paramagnetic iron oxide nanoparticles (SPIONs) comprises a dextran with an average molecular weight of 5000, sodium periodate, potassium cyanide, poly-L-histidine with a molecular weight of 5000-25000, and poly (ethylene oxide), 6-arm anthracenes with an average molecular weight of 12000, 90% oleic acid, 1-octadecene, oleyl alcohol and n-hexane.

According to one embodiment herein, the synthesis of the super paramagnetic iron oxide nanoparticles (SPIONs) is carried out in the following sequences. At first the carboxylated-dextran is prepared. Secondly, the super paramagnetic iron oxide nanoparticles (SPIONs) are prepared. Thirdly, the super para magnetic iron oxide nanoparticles (SPIONs) are coated with carboxyl-dextran. Then the super paramagnetic iron oxide nanoparticles (SPIONs) coated with carboxyl-dextran coating are further subjected to gold coating Finally, the gold coated, fluorescent gap bearing the super paramagnetic iron oxide nanoparticles (SPIONs) are characterized.

A standard protocol is followed for the preparation of the carboxylated-dextran. For the preparation of carboxylated-dextran, the hydroxyl groups in the dextran are oxidized to aldehyde groups by sodium periodate. Further, sodium periodate is dissolved in the de-oxygenated distilled water and introduced to dextran. An amount of 4 gm of sodium periodate is dissolved in 30 ml of de-oxygenated distilled water. The obtained solution is homogenized for 2 hrs at a room temperature and the homogenized solution is dialyzed using a membrane bag with a cut-off molecular weight of 1,000 for 4 days. Then the obtained solution is subjected to potassium cyanide for the preparation of intermediate cyanohydrins. Finally, the carboxylic acid group is created on the terminal units of dextran by the hydrolysis of the obtained intermediate cyanohydrins. Further, the prepared carboxylated dextran is lyophilized and stored at −80° C.

According to one embodiment herein, a polyol route is employed to obtain the nano particles with a narrow size distribution. The method of preparation of SPIONs involves the following sequences. The iron-oleate complexes are prepared by reacting sodium oleate and iron (III) chloride. For the synthesis of SPIONs with a particle size of 13 nm, 18 gm (20 mmol) of iron oleate complex and 5.7 gm of oleic acid (20 mmol) are dissolved in 100 gm of 1-octadecene at room temperature to obtain a reaction mixture. The reaction mixture is degassed at 80° C. temperature for 2 hrs. The reaction mixture is heated to a reflex temperature at a heating rate of 3° C./min. The reaction mixture is then kept for 30 min under inert atmosphere. After the reaction, the reaction vessel is repeatedly cooled at room temperature and 500 mL of acetone is added to precipitate the SPIONs. The SPIONs are separated by the centrifugation and dispersed in hexane.

According to one embodiment herein, the super paramagnetic iron oxide nanoparticles (SPIONs) are coated with carboxyl-dextran. The ligand exchange process is used for coating the prepared hydrophobic nanoparticles with carboxyl-dextran. The SPIONs with an iron concentration of 1 mg/ml is prepared and mixed with dextran ligands in a di-methyl sulfoxide (DMSO). The DMSO is a dipolar solvent and the reactions of nano particles and polymers are carried out at room temperature for 72 hrs, while shaking with a shaking incubator. The DMSO is used to form a homogenous solution with both aqueous polymer solution and organic solvent. Specifically, 1 ml of the stock SPION solution is mixed with dextran, in 30 ml of DMSO. After the completion of the reaction, the SPIONs are magnetically collected by a strong magnetic field using a magnetic activated cell sorter (MACS™) system. Further the collected SPIONs are dispersed into 1 ml of distilled or deionized (DI) water. The water soluble SPIONs are completely stable at room temperature.

According to one embodiment herein, the super paramagnetic iron oxide nanoparticles (SPIONs) coated with carboxyl-dextran coating are further subjected to gold coating. The SPIONs are subjected to gold coating to create a fluorescent polymeric gap. The SPIONs are coated with gold to get a smooth surface. The smooth gold-shell SPIONs are prepared by mixing the carboxyl-dextran coated SPIONs with poly (ethylene oxide) and 6-arm anthracene terminated in distilled water using shaking incubator for 10 hrs. The resultant materials after mixing are collected with strong magnet and washed with distilled water for several times. Poly-L histinde (PLH) is added to the solution of SPIONs and the pH of the solution is adjusted to be in the range of 5-6, using 0.1N hydrochloric acid (HCl). After incubating for 60 min, the magnetic nanoparticles are collected with a magnet and washed for several times with distilled water. The obtained solution is mixed with HAuCl₄ (w/w 1%), for 20 min. The pH of the solution is adjusted to be in the range of 9-10 with sodium hydroxide (NaOH). The NH₂OH and HCl is added to the solution and mixed well till the color of the colloidal suspension turns to a dark blue color. The solution is washed for several times, redispersed in distilled water using sonicator and kept at 2-8° C.

According to one embodiment herein, the gold coated, fluorescent gap bearing super paramagnetic iron oxide nanoparticles (SPIONs) are characterized. The first method of characterization is dynamic light scattering (DLS) measurement. The DLS measurement is conducted with a Malvern PCS-4700 instrument equipped with a 256-channel correlator. The 488.0 nm line of a Coherent Innova-70 Ar ion laser is used as the incident beam. The power of the laser used is 250 mW. The scattering angle 0, employed is in the range of 40°-140°. The temperature is maintained at 25° C. with an external circulator. Further, the data obtained is subjected to data analysis and interpretation. Data analysis is performed according to standard procedures, and interpreted through a cumulated expansion of the field auto correlation function of the second order. A constrained regularization method CONTIN is applied to invert the experimental data to obtain a distribution of the decay rates. The size and shape of the nanoparticles are evaluated using a Phillips CM200 transmission electron microscope (TEM) equipped with an AMT 2×2 CCD camera with an accelerating voltage of 200 kV. The sample for TEM is prepared by placing and drying a drop of the suspension on a copper grid.

According to one embodiment herein, the fluorescent gap bearing super paramagnetic iron oxide nanoparticles (SPIONs) are further subjected to magnetization measurements. The solid dry powder of the SPION sample is taken and subjected to Quantum Design Superconducting Quantum Interference Device (SQUID) MPMS-XL7 magnetometer. A hysteresis experiment is performed in the range of −5T≦H≦+5T at T=300K. The in vitro MRI experiments are performed at 8.5 MHz using a 0.2 Tesla Artoscan Imager by Esaote S.p.A. A Spin Echo (SE) T₂ pulse sequence with the imaging parameters of TR/TE/NEX=2000 ms/80 ms/1, a matrix=256*192, and a FOV=180*180 is used.

According to one embodiment herein, the analysis of super paramagnetic iron oxide nanoparticles (SPIONs) exhibit multimodal imaging. The gold shell bearing SPIONs are compared with the bare SPIONs. The results reveal that the smooth, gold shell coated SPIONs possess both strong scattering property of gold nanoshell and fluorescence capability of polymeric gap. Both the properties make the gold coated and fluorescent gap bearing SPIONs as a useful dual-optical imaging probe.

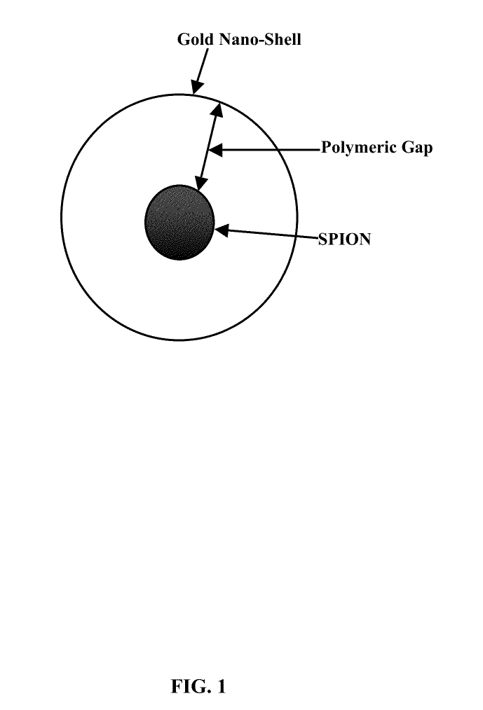

FIG. 1 illustrates a schematic diagram indicating a structure of gold coated super paramagnetic iron oxide nanoparticles (SPIONs), with fluorescent polymer dye, according to one embodiment herein. The gold coated SPION has three zones. The innermost zone of the SPION represents the core. The outermost zone the SPION is the gold nano-shell. The gap between the core and shell of SPIONs comprise of a polymer. The polymer is a fluorescent dye. A fluorescent polymeric dye is sandwiched in a gap between the inner most core and the outermost gold shell.

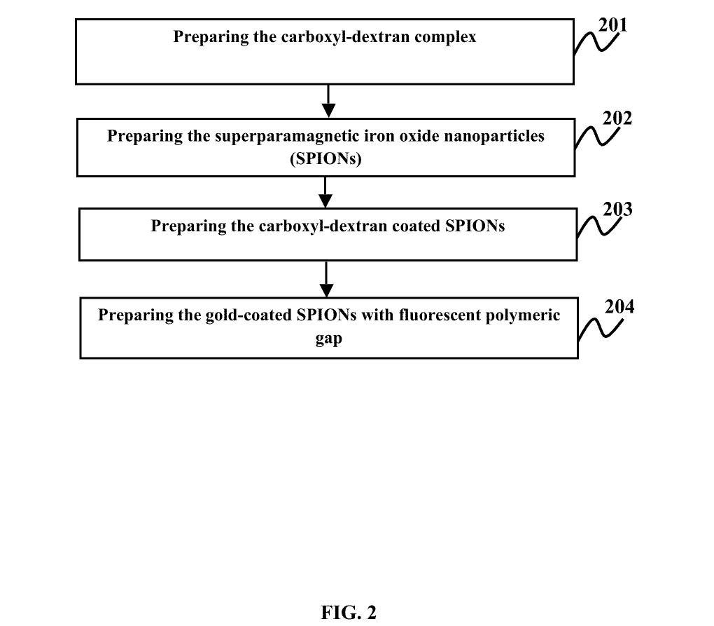

FIG. 2 illustrates a flow chart explaining a method of synthesizing the super paramagnetic iron oxide nanoparticles (SPIONs), with fluorescence polymeric dye, according to one embodiment herein. The first step in the synthesis of SPIONs is the preparation of the carboxyl-dextran (101). A standard protocol is followed for the preparation of the carboxylated-dextran. For the preparation of carboxylated-dextran, the hydroxyl groups in the dextran are oxidized to aldehyde groups by sodium periodate. Further, sodium periodate is dissolved in the de-oxygenated distilled water and introduced to dextran solution (4 gm in 30 ml of de-oxygenated distilled water). The obtained solution is homogenized for 2 hrs at room temperature followed by dialyzing with membrane bag with a 1,000 cut-off molecular weight for 4 days. The next step is that the obtained solution is subjected to potassium cyanide for the preparation of intermediate cyanohydrins. Finally, the carboxylic acid group is created on the terminal units of dextran by the hydrolysis of the obtained intermediate cyanohydrins. Further, the prepared carboxylated dextran is lyophilized and stored at −80° C.

The second step is the preparation of super paramagnetic iron oxide nanoparticles (SPIONs) (102). First, iron-oleate complexes are prepared by reacting sodium oleate and iron (III) chloride. For the synthesis of SPIONs with a particle size of 13 nm, 18 gm (20 mmol) of iron oleate complex and 5.7 gm of oleic acid (20 mmol) are dissolved in 100 gm of 1-octadecene at room temperature to obtain a reaction mixture. Further the reaction mixture is degassed at 80° C. temperature for 2 hrs. The reaction mixture is heated to a reflex temperature at a heating rate of 3° C./min. The reaction mixture is then kept for 30 min under the inert atmosphere. After the reaction, the reaction vessel is repeatedly cooled at room temperature and 500 mL of acetone is added to precipitate the SPIONs. The SPIONs are separated by the centrifugation and dispersed in hexane.

The third step in the preparation of super paramagnetic iron oxide nanoparticles (SPIONs) is the preparation of carboxyl-dextran coated SPIONs (103). The first step in coating is to mix SPIONs with an iron concentration of 1 mg/ml with dextran ligands in a di-methyl sulfoxide (DMSO). The DMSO is a dipolar solvent and the reactions of nanoparticles and polymers are conducted at room temperature for 72 hrs, in a shaking incubator. The DMSO forms a homogenous solution with both aqueous polymer solution and organic solvent. Specifically, 1 ml of the stock SPION solution is mixed with dextran, in 30 ml of DMSO. After the reaction, the SPIONs are magnetically collected by a strong magnetic field. The strong magnetic field is provided by magnetic activated cell sorter (MACS™) system. Further the SPIONs are dispersed into 1 ml of distilled water. The water soluble SPIONs are completely stable at room temperature.

The fourth step in the preparation of super paramagnetic iron oxide nanoparticles (SPIONs) is the preparation of gold-coated SPIONs with fluorescent polymeric gap (104). The SPIONs are subjected to gold coating to create a fluorescent polymeric gap. The SPIONs are coated with gold to get a smooth surface. The smooth gold-shell SPIONs are prepared by mixing the carboxyl-dextran coated SPIONs with poly (ethylene oxide) for 10 hrs in the presence of 6-arm anthracene terminated in distilled water using shaking incubator. After 10 hrs the materials are collected with a strong magnet and washed several times with distilled water. Poly-L histinde (PLH) is added to the solution of SPIONs and the pH is adjusted to be in the range of 5-6, using 0.1N hydrochloric acid (HCl). After incubating for 60 min, the magnetic nano particles are collected with a magnet and washed several times with distilled water. The obtained solution is mixed with HAuCl₄ (w/w 1%), for 20 min. The pH is adjusted to be in the range of 9-10 with sodium hydroxide (NaOH). Further NH₂OH and HCl is added to the solution and mixed well till the color of the colloidal suspension turns to a dark blue color. The solution is washed several times, and redispersed in distilled water using sonicator. The temperature is then maintained between 2-8° C.

The gold coated, fluorescent gap bearing super paramagnetic iron oxide nanoparticles (SPIONs) are subjected to characterization. The first method of characterization is dynamic light scattering (DLS) measurement. The DLS measurement is conducted with a Malvern PCS-4700 instrument equipped with a 256-channel correlator. The 488.0 nm line of a Coherent Innova-70 Ar ion laser is used as the incident beam. The power of the laser is 250 mW. The scattering angle θ, is in the range of 40°-140°. The temperature is maintained at 25° C. with an external circulator. Further the data obtained is subjected to analysis and interpretation. The data analysis and interpretation is done through a cumulative expansion of the field autocorrelation function to the second order. Further, a constrained regularization method such as CONTIN is applied to invert the experimental data to obtain a distribution of the decay rates. The size and shape of the nanoparticles is evaluated using a Phillips CM200 transmission electron microscope (TEM) equipped with an AMT 2×2 CCD camera with an accelerating voltage of 200 kV. The sample for TEM is prepared by placing a drop of the suspension on a copper grid and drying the suspension. Following table illustrates the results of DLS characterization:

| Nanoparticles | D_{H} | PDI^{b} | <D_{H}> (nm)^{c} |
|---|---|---|---|
| Bare Nanoparticles | 13.5 ± 0.1 | 0.089 | 14.8 ± 0.2 |
| Smooth gold coated SPIONs | 21.1 ± 0.5 | 0.102 | 22.4 ± 0.7 |

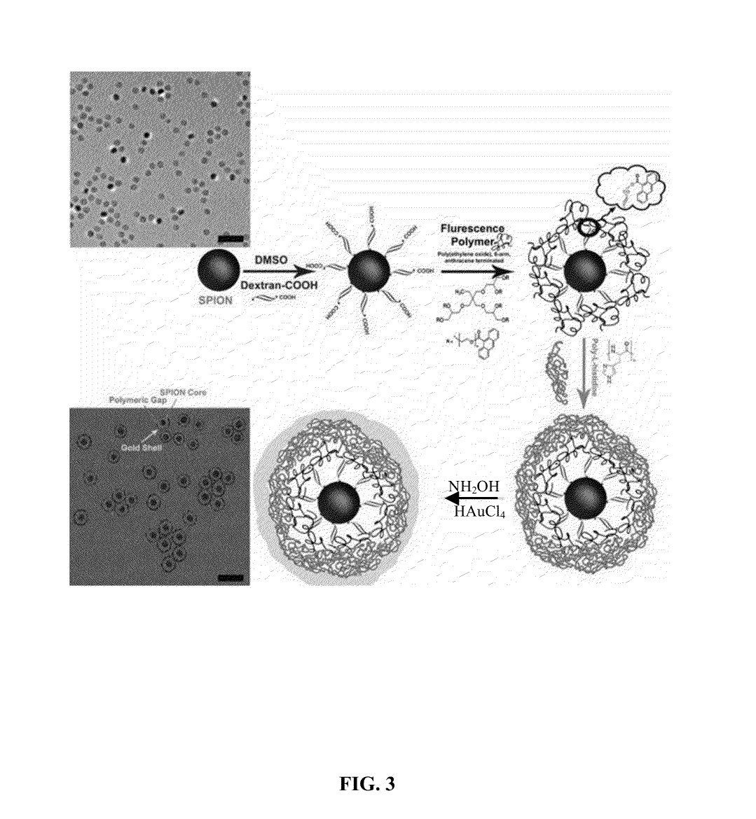

FIG. 3 illustrates a schematic diagram indicating the different steps in the synthesis of super paramagnetic iron oxide nanoparticles (SPIONs) with fluorescent polymeric gap, according to one embodiment herein. FIG. 3 summarizes the steps followed for the preparation of the carboxyl-dextran, preparation of SPIONs, preparation of carboxyl-dextran coated SPIONs in presence of dimethyl sulfoxide (DMSO) and preparation of gold-coated SPIONs with fluorescent polymeric gap. The fluorescent polymeric gap comprises 6-arm anthracene terminated. The SPION core and fluorescent gap is coated with chloroauric acid (HAuCl₄) in presence of ammonium hydroxide.

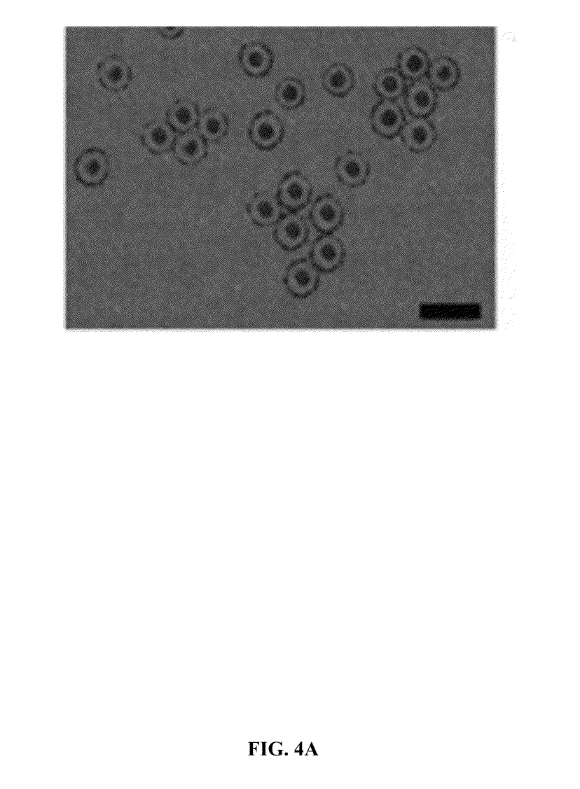

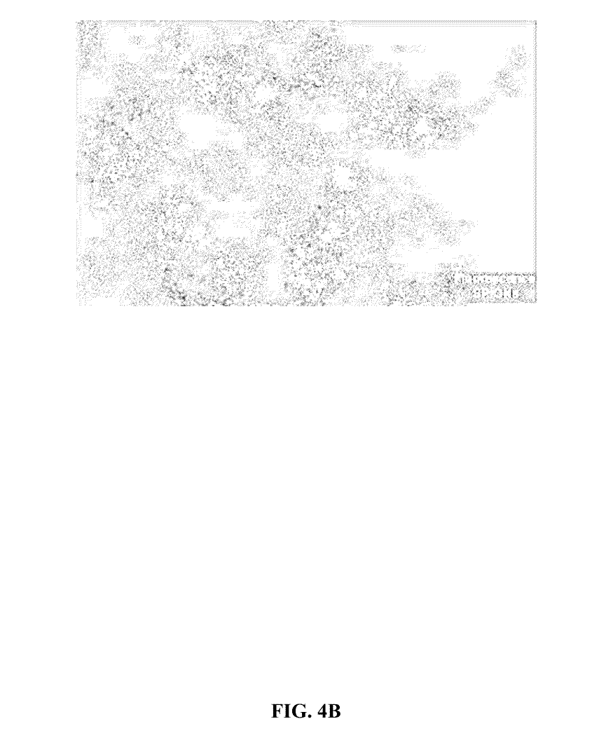

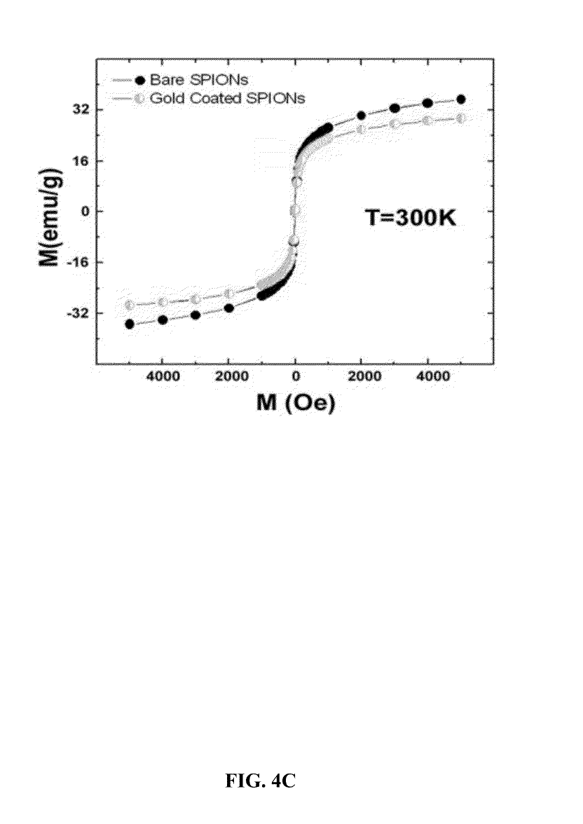

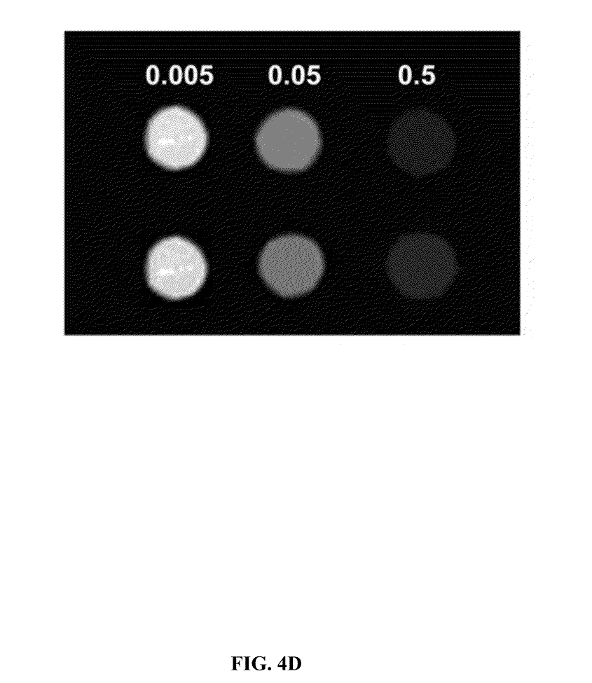

FIG. 4A illustrates a transmission electron microscope (TEM) image indicating a dark field imaging of bare SPIONs, according to an embodiment herein. In order to show the capability of the engineered nanoparticles for multimodality imaging, the bare SPIONs and smooth-shaped gold shell coated SPIONs, with fluorescence polymeric gap, are tested with conventional modalities, including dark field imaging as shown in FIG. 4A. According to the results, it is seen that both the strong scattering property of gold nanoshell and fluorescence capability of polymeric gap makes the smooth shaped gold shell coated SPIONs an excellent dual-optical imaging probe. More specifically, dilute bare SPIONs and smooth-shaped gold shell coated SPIONs are spread on glass cover slips, resulting in spatially isolated single nanoparticles on the surface. Under dark field imaging conditions, the bare particles do not have detectable effects whereas gold shell coated SPIONs are easily detectable by both gold shell and fluorescence dye. The major advantage of the gold coated SPIONS with fluorescent dye particles is that the scattering-based imaging by gold shell does not allow deep tissue imaging, but fluorescence capability and MRI does.

FIG. 4B illustrates a transmission electron microscope (TEM) image indicating a dark field imaging obtained with smooth gold coated SPIONS with fluorescent dye, according to one embodiment herein. In order to show the capability of the engineered nanoparticles for multimodality imaging, the bare SPIONs and smooth-shaped gold shell coated SPIONs, with fluorescence polymeric gap, are tested with conventional modalities, including dark field imaging as shown in FIG. 4B. According to the results, it is seen that both the strong scattering property of gold nanoshell and fluorescence capability of polymeric gap makes the smooth shaped gold shell coated SPIONs an excellent dual-optical imaging probe. More specifically, dilute bare SPIONs and smooth-shaped gold shell coated SPIONs are spread on glass cover slips, resulting in spatially isolated single nanoparticles on the surface. Under dark field imaging conditions, bare particles do not have detectable effects whereas gold shell coated SPIONs are easily detectable by both gold shell and fluorescence dye. The major advantage of the gold coated SPIONS with fluorescent dye particles is that the scattering-based imaging by gold shell does not allow deep tissue imaging, but fluorescence capability and MRI does.

FIG. 4B indicates the merged fluorescent and scattering image wherein the grey spots are created by the scattering of gold shell. Further the dark grey spot in the image corresponds to the fluorescence trapped polymer between the core and the shell of the nanoparticle.

FIG. 4C illustrates a graph indicating the magnetization of for bare superparamagnetic iron oxide nanoparticles (SPIONs) and smooth gold shaped SPIONS with fluorescent dye with respect to different magnetic field conditions at 300 K, according to the embodiments herein. The plots are obtained after subjecting the smooth and bare SPIONs to Superconducting Quantum Interference Device (SQUID) for characterization. The solid dry powder of the SPION sample is taken and subjected to Quantum Design Superconducting Quantum Interference Device (SQUID) MPMS-XL7 magnetometer. A hysteresis experiment is performed in the range of −5T≦H≦+5T at T=300K. The in vitro MRI experiments are performed at 8.5 MHz using a 0.2 Tesla Artoscan Imager by Esaote S.p.A. A Spin Echo (SE) T₂ pulse sequence with TR/TE/NEX=2000 ms/80 ms/1, a matrix=256*192, and a FOV=180*180 are taken as imaging parameters. The plot is created at 300K for bare SPIONs and smooth shaped gold coated SPIONs. The graphical plots in FIG. 4C illustrates that the magnetic properties of the bare SPIONs are maintained after being coated with fluorescence polymer and with the thin gold shells.

FIG. 4D illustrates a magnetic resonance imaging (MRI) of bare super paramagnetic iron oxide nanoparticles (SPIONs) and smooth gold coated SPIONS with fluorescent dye, with different ionic concentrations, according to the embodiments herein. The image illustrates the SPIONs with different iron concentrations in the core. FIG. 4D indicates different dilutions of the iron i.e. 0.005, 0.05 and 0.5 mg/ml and their effect on the image contrasting capability. FIG. 4D illustrates that the SPIONs with different dilutions exhibit identical image contrast irrespective of the iron concentration in the core of SPIONs.

In summary, a new class of SPIONs-gold core-shell NPs has been developed. In contrast to the previous arts in which gold shells are deposited directly on SPIONs, the core and shell of the gold core-shell SPIONS with fluorescent polymer dye are spatially separated with a dielectric polymer layer. Using fluorescence polymer in the gap between gold shell and SPION core, new stable fluorescence modal was added in addition to the other reported properties including electronic, magnetic, optical, acoustic and thermal responses, which allow multimodality imaging. It is seen that the prepared jagged gold surface also allows simple conjugation with various type of biomolecular through thiol binding to enhance all-in-one nanoprobe for non-invasive imaging, molecular theranosis of complex diseases. In addition, several polymers with great fluorescence properties which could not be used, due to their non-biocompatible properties, can be used in this new nanoprobe.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A plasmonic stable fluorescence super paramagnetic iron oxide nanoparticle (SPION) comprises:
a nano metal core, and wherein the nano metal core is formed with a SPION;
a nano shell arranged around the nano metal core, and wherein the nano shell is a gold shell;
a dielectric polymer layer formed in a gap between the nano metal core and the nano shell, and wherein the dielectric polymer layer is a fluorescence polymer layer.

2. The plasmonic stable fluorescence super paramagnetic iron oxide nanoparticle (SPION) according to claim 1, wherein the nano metal core is made up of ferrous chloride.

3. The plasmonic stable fluorescence super paramagnetic iron oxide nanoparticles (SPION) according to claim 1, wherein the fluorescent polymer is 6-arm anthracene terminated.

4. The plasmonic stable fluorescence super paramagnetic iron oxide nanoparticles (SPIONs) according to claim 1, wherein the SPION has a particle size of 13 nm.

5. A method of synthesizing plasmonic stable fluorescence super paramagnetic iron oxide nanoparticles (SPIONs), the method comprises the steps of:
preparing carboxylated dextran;
preparing super paramagnetic iron oxide nanoparticle (SPION);
preparing carboxylated dextran coated SPION; and
preparing a gold coated SPION with fluorescence polymeric gap.

6. The method according to claim 5, wherein the step of preparing carboxyl-dextran comprises:
dissolving sodium periodate in deoxygenated distilled water and wherein an amount sodium periodate dissolved in deoxygenated distilled water is 4 gm, and wherein an amount of deoxygenated distilled water used for dissolving 4 gm sodium periodate is 30 ml;
adding dextran solution in the solution of sodium periodate;
homogenizing the solution of periodate added with dextrin for 2 hrs at room temperature;
dialyzing the homogenized solution in a membrane bag for 4 days, and wherein the membrane bag has a cut-off molecular weight of 1,000;
preparing a cyanohydrin intermediate by interacting the dialyzed solution with potassium cyanide;
obtaining a carboxylated dextran by a hydrolysis of the intermediate cyanohydrins;
lyophilizing the carboxylated dextran at −80° C.; and
storing the carboxylated dextran which is lyophilized.

7. The method according to claim 5, wherein the step of preparing SPION comprises:
dissolving iron oleate complex and 1-octadecene in oleic acid at room temperature to obtain a reaction mixture, wherein an amount of iron oleate complex dissolved is 18 gm, and wherein an amount of iron oleic acid used for dissolving is 5.7 gm, and wherein an amount of 1-octadecene dissolved is 100 gm, and wherein a molarity of the reaction mixture is 20 mmol;
degassing the reaction mixture at 80° C. for 2 hrs;
heating the reaction mixture to a reflux temperature at a rate of 3° C./min;
incubating the reaction mixture for 30 min under an inert atmosphere;
rapidly cooling the reaction mixture to room temperature;
adding 500 ml of acetone to the cooled reaction mixture;
precipitating the SPIONs;
separating the SPIONs with a concentration of 1 mg/ml by centrifugation; and
dispersing the SPIONs in hexane.

8. The method according to claim 5, wherein the step of synthesizing the carboxyl-dextran coated SPIONs comprises:
mixing SPION stock solution with dextran, in dimethyl sulfoxide (DMSO), and wherein an amount of SPION stock solution mixed with dimethyl sulfoxide (DMSO) is 1 ml, and wherein an amount of dimethyl sulfoxide (DMSO) mixed with SPION stock solution is 30 ml;
magnetically collecting the SPIONs through a strong magnetic field using magnetically activated cell sorter (MACS®) system; and
redispersing the collected SPIONs into 1 ml of distilled water.

9. The method according to claim 5, wherein the step of synthesizing the gold coated SPIONs with fluorescent polymeric gap comprises:
mixing carboxyl-dextran coated SPIONs with poly(ethylene oxide) for 10 hrs and 6-arm anthracene terminated in distilled water using shaking incubator;
collecting coated SPIONs with strong magnet;
adding poly-L histidine to a solution of the SPIONs;
adjusting a pH of a solution of SPIONS and poly-L histidine using 0.1N HCl, and wherein the pH of the solution of SPIONS is adjusted to be within 5-6;
incubating the pH adjusted solution of SPIONs for 60 min;
collecting magnetic SPIONS using a magnet after incubating the SPIONs for 60 minutes;
washing the incubated SPIONS for several times with distilled water;
mixing a solution of incubated SPIONS with HAuCl₄ (w/w 1%), for 20 min;
adjusting a pH of incubated SPIONS solution mixed with HAuCl₄, to be in the range of 9-10 using NaOH;
adding solution of NH₂OH HCl to the solution of SPIONS and mixing the solution of SPIONS to obtain a colloidal suspension, wherein NH₂OH HCl is added to the solution of SPIONS till a color of the colloidal suspension turns to dark blue color;
washing the colloidal suspension several times with distilled water and suspending a colloid in distilled water;
incubating the colloid in a sonicator at 2-8° C.
